(19) United States
(12) Patent Application Publication
(10) Pub. No.: US 2025/0188535 A1
(43) Pub. Date: Jun. 12, 2025

(54) SPATIAL-SEQUENCING METHODS

(71) Applicant: THE UNIVERSITY OF BRITISH COLUMBIA, Vancouver (CA)

(72) Inventors: Geoffrey SCHIEBINGER, Vancouver (CA); Nozomu YACHIE, Vancouver (CA); Matthieu HEITZ, Vancouver (CA); Anton AFANASSIEV, Vancouver (CA); Yusuke KIJIMA, Vancouver (CA); Laura GREENSTREET, Ithaca, NY (US)

(73) Assignee: THE UNIVERSITY OF BRITISH COLUMBIA, Vancouver, BC (CA)

(21) Appl. No.: 18/841,129

(22) PCT Filed: Feb. 24, 2023
(86) PCT No.: PCT/CA2023/050239

§ 371 Date: Aug. 23, 2024

Related U.S. Application Data
(60) Provisional application No. 63/322,386, filed on Mar. 22, 2022.
(60) Provisional application No. 63/314,089, filed on Feb. 25, 2022.

(51) Int. Cl. C12Q 1/6869

(52) U.S. Cl. CPC C12Q 1/6869

(57) ABSTRACT

Spatial-sequencing methods are disclosed.

### CROSS-REFERENCE TO RELATED APPLICATIONS

This application claims the benefit of and priority to U.S. provisional patent application No. 63/314,089 filed on Feb. 25, 2022 and U.S. provisional patent application No. 63/322,386 filed on Mar. 22, 2022. The entire contents of U.S. provisional patent application Nos. 63/314,089 and 63/322,386 are incorporated by reference herein.

### FIELD

This disclosure relates generally to spatial-sequencing methods.

### RELATED ART

Known spatial-sequencing methods may be limited.

### SUMMARY

According to at least one embodiment, there is disclosed a spatial-sequencing method comprising: causing a plurality of analytes from at least one sample to become associated with, at least, respective location-specific markers of a plurality of location-specific markers; and causing a plurality of multiple-location markers to become associated with, at least, respective location-specific markers of the plurality of location-specific markers; wherein each analyte of the plurality of analytes is of a respective analyte type of a plurality of different analyte types; wherein each location-specific marker of the plurality of location-specific markers is associated with a respective location of a plurality of different locations relative to the sample; wherein each location-specific marker of the plurality of location-specific markers is of a respective location-specific-marker type, associated with the location associated with the location-specific marker, of a plurality of different location-specific-marker types; wherein each multiple-location marker of the plurality of multiple-location markers is of a respective multiple-location-marker type of a plurality of different multiple-location-marker types; and wherein causing the plurality of multiple-location markers to become associated with the respective location-specific markers comprises, for each multiple-location-marker type of the plurality of different multiple-location-marker types, causing multiple-location markers of the plurality of multiple-location markers and of the multiple-location-marker type to become associated with the respective location-specific markers associated with multiple locations of the plurality of the locations and associated with the multiple-location-marker type.

In some embodiments, causing the plurality of analytes to become associated with the respective location-specific markers comprises causing the plurality of analytes to bind to the respective location-specific markers.

In some embodiments, causing the plurality of multiple-location markers to become associated with the respective location-specific markers comprises causing the plurality of multiple-location markers to bind to the respective location-specific markers.

In some embodiments, for each multiple-location-marker type of the plurality of different multiple-location-marker types, the multiple locations of the plurality of the locations and associated with the multiple-location-marker type are within a region associated with the multiple-location-marker type.

In some embodiments, at least some analytes of the plurality of analytes comprise respective polymeric molecules from the at least one sample.

In some embodiments, at least some of the polymeric molecules of the plurality of analytes have respective nucleotide sequences.

In some embodiments, at least some of the polymeric molecules of the plurality of analytes comprise respective messenger ribonucleic acid (mRNA) molecules.

In some embodiments, at least some of the polymeric molecules of the plurality of analytes comprise respective ribonucleic acid (RNA) molecules.

In some embodiments, at least some of the polymeric molecules of the plurality of analytes comprise respective deoxyribonucleic acid (DNA) molecules.

In some embodiments, at least some of the DNA molecules of the plurality of analytes comprise respective open chromatin regions.

In some embodiments, at least some of the plurality of different analyte types are respective different nucleotide sequences of at least some of the open chromatin regions of the at least some of the DNA molecules of the plurality of analytes.

In some embodiments, at least some of the plurality of different analyte types are respective different methylation patterns of at least some of the DNA molecules of the plurality of analytes.

In some embodiments, at least some of the plurality of different analyte types are respective different contact points of at least some of the DNA molecules of the plurality of analytes.

In some embodiments, at least some of the plurality of different analyte types are respective different nucleotide sequences of at least some of the polymeric molecules of the plurality of analytes.

In some embodiments, at least some analytes of the plurality of analytes comprise respective proteins.

In some embodiments, at least some of the plurality of different analyte types are respective different protein types.

In some embodiments, each analyte of the plurality of analytes comprises a respective polymeric molecule from the at least one sample.

In some embodiments, each analyte of the plurality of analytes has a respective nucleotide sequence.

In some embodiments, each analyte of the plurality of analytes comprises a respective mRNA molecule.

In some embodiments, the at least one sample comprises at least one biological sample.

In some embodiments, the at least one biological sample comprises animal tissue.

In some embodiments, the at least one biological sample comprises plant material.

In some embodiments, the at least one biological sample comprises fungal material.

In some embodiments, the at least one biological sample comprises a bacterial distribution.

In some embodiments, the at least one sample is already cryogenically frozen when the plurality of analytes become associated with the respective location-specific markers.

In some embodiments, the at least one sample is already paraffin-embedded when the plurality of analytes become associated with the respective location-specific markers.

In some embodiments, each location-specific marker of the plurality of location-specific markers comprises a nucleotide sequence, and the plurality of different location-specific-marker types is a plurality of different nucleotide sequences.

In some embodiments, each multiple-location marker of the plurality of multiple-location markers comprises a nucleotide sequence, and the plurality of different multiple-location-marker types is a plurality of different nucleotide sequences.

In some embodiments, for each location of the plurality of different locations, each location-specific marker of the plurality of location-specific markers and associated with the location is of a same location-specific-marker type, associated with the location, of the plurality of different location-specific-marker types.

In some embodiments, causing the plurality of multiple-location markers to become associated with the respective location-specific markers comprises, for each multiple-location-marker type of the plurality of different multiple-location-marker types, and for each location of the plurality of the locations and associated with the multiple-location-marker type, causing the multiple-location markers of the plurality of multiple-location markers and of the multiple-location-marker type to become associated with the respective location-specific markers at the location in a respective abundance that is positively correlated with a relative proximity of the location to a reference location of the multiple-location-marker type.

In some embodiments, for each multiple-location-marker type of the plurality of different multiple-location-marker types, the reference location of the multiple-location-marker type is a source of the multiple-location markers of the plurality of multiple-location markers and of the multiple-location-marker type.

In some embodiments, the method further comprises causing the plurality of multiple-location markers to be created.

In some embodiments, causing the plurality of multiple-location markers to be created comprises causing organisms to create the plurality of multiple-location markers.

In some embodiments, the organisms comprise bacteria.

In some embodiments, the bacteria comprise Escherichia coli.

In some embodiments, causing the plurality of multiple-location markers to be created comprises causing multiple-location-marker-creating enzymes to create the plurality of multiple-location markers.

In some embodiments, the multiple-location-marker-creating enzymes comprise polymerases.

In some embodiments, causing the multiple-location-marker-creating enzymes to create the plurality of multiple-location markers comprises, for each multiple-location-marker type of the plurality of different multiple-location-marker types, causing the multiple-location-marker-creating enzymes to create multiple-location markers of the multiple-location-marker type from a multiple-location-marker-creating molecule comprising a nucleotide sequence.

In some embodiments, when the plurality of analytes become associated with the respective location-specific markers, and when the plurality of multiple-location markers become associated with the respective location-specific markers, each location-specific marker of the plurality of location-specific markers is bound to a respective body of a plurality of bodies, each body of the plurality of bodies associated with a respective different location of the plurality of the locations.

In some embodiments, each body of the plurality of bodies comprises a bead.

In some embodiments, the method further comprises, for each location-specific-marker type of the plurality of different location-specific-marker types, causing a source of the location-specific markers of the plurality of location-specific markers and of the location-specific-marker type to create the location-specific markers of the plurality of location-specific markers and of the location-specific-marker type.

In some embodiments, for each location-specific-marker type of the plurality of different location-specific-marker types, the source of the location-specific markers of the plurality of location-specific markers and of the location-specific-marker type comprises a location-specific-marker-creating molecule, and causing the source of the location-specific markers of the plurality of location-specific markers and of the location-specific-marker type to create the location-specific markers of the plurality of location-specific markers and of the location-specific-marker type comprises causing location-specific-marker-creating enzyme to amplify the location-specific-marker-creating molecule.

In some embodiments, for each location-specific-marker type of the plurality of different location-specific-marker types, the location-specific-marker-creating molecule comprises a nucleotide sequence.

In some embodiments, for each location-specific-marker type of the plurality of different location-specific-marker types, the location-specific-marker-creating enzyme comprises a polymerase.

In some embodiments, when the plurality of analytes become associated with the respective location-specific markers, and when the plurality of multiple-location markers become associated with the respective location-specific markers, each location-specific marker of the plurality of location-specific markers is suspended in a gel.

In some embodiments, when the plurality of analytes become associated with the respective location-specific markers, and when the plurality of multiple-location markers become associated with the respective location-specific markers, each multiple-location marker of the plurality of multiple-location markers is suspended in a gel.

In some embodiments, when the plurality of analytes become associated with the respective location-specific markers, and when the plurality of multiple-location markers become associated with the respective location-specific markers, each multiple-location marker of the plurality of multiple-location markers is suspended in the gel.

In some embodiments, when an analyte of the plurality of analytes becomes associated with a location-specific marker of the plurality of location-specific markers, the analyte is at the location associated with the location-specific-marker type of the location-specific marker.

In some embodiments, when the plurality of multiple-location markers become associated with the respective location-specific markers, each location-specific marker of the plurality of location-specific markers is at the respective location of the plurality of different locations.

In some embodiments, when the plurality of analytes become associated with the respective location-specific markers, each location-specific marker of the plurality of location-specific markers is at the respective location of the plurality of different locations.

In some embodiments, each location of the plurality of different locations is a respective location in the at least one sample.

In some embodiments, each location of the plurality of different locations is a respective location on at least one surface of the at least one sample.

In some embodiments: the sample comprises a plurality of sample components; and the method further comprises detaching the plurality of sample components such that each sample component of the plurality of sample components is detached from each other sample component of the plurality of sample components.

In some embodiments, the plurality of analytes become associated with the respective location-specific markers of the plurality of location-specific markers after each sample component of the plurality of sample components is detached from each other sample component of the plurality of sample components.

In some embodiments, the plurality of multiple-location markers become associated with the respective location-specific markers of the plurality of location-specific markers after each sample component of the plurality of sample components is detached from each other sample component of the plurality of sample components.

In some embodiments, each sample component of the plurality of sample components comprises a respective different cell of the at least one sample.

In some embodiments, each location of the plurality of different locations is a respective location of a respective one of the plurality of sample components in the at least one sample before detaching the plurality of sample components.

In some embodiments, causing the plurality of multiple-location markers to become associated with the respective location-specific markers comprises causing the plurality of multiple-location markers to be transferred from at least one surface to the respective location-specific markers of the plurality of location-specific markers.

In some embodiments, causing the plurality of multiple-location markers to become associated with the respective location-specific markers comprises, for each multiple-location-marker type of the plurality of different multiple-location-marker types, causing the multiple-location markers of the plurality of multiple-location markers and of the multiple-location-marker type to be transferred from at least one surface to the multiple locations of the plurality of the locations and associated with the multiple-location-marker type.

In some embodiments, causing the plurality of multiple-location markers to become associated with the respective location-specific markers comprises, for each multiple-location-marker type of the plurality of different multiple-location-marker types, causing the multiple-location markers of the plurality of multiple-location markers and of the multiple-location-marker type to propagate, from a source of the multiple-location markers of the plurality of multiple-location markers and of the multiple-location-marker type, to the multiple locations of the plurality of the locations and associated with the multiple-location-marker type.

In some embodiments, the method further comprises determining, at least, a multiple-location-marker estimation, for each location-specific-marker type of the plurality of different location-specific-marker types and for each multiple-location-marker type of the plurality of different multiple-location-marker types, of an estimated number of location-specific markers, of the plurality of location-specific markers and of the location-specific-marker type, associated with respective multiple-location markers of the plurality of multiple-location markers and of the multiple-location-marker type.

In some embodiments, the method further comprises, in response to at least the multiple-location-marker estimations, identifying a position of the respective location associated with each location-specific-marker type of the plurality of different location-specific-marker types.

In some embodiments, the method further comprises identifying a position of the respective location associated with each location-specific-marker type of the plurality of different location-specific-marker types.

In some embodiments, identifying the position of the respective location associated with each location-specific-marker type of the plurality of different location-specific-marker types comprises dimensionality reduction.

In some embodiments: identifying the position of the respective location associated with each location-specific-marker type of the plurality of different location-specific-marker types comprises dimensionality reduction; and the dimensionality reduction comprises, for each location-specific-marker type, identifying at least a manifold embedded in the estimated numbers of location-specific markers of the plurality of location-specific markers and of the location-specific-marker type.

In some embodiments, the manifold is a three-dimensional manifold.

In some embodiments, the manifold is a two-dimensional manifold.

In some embodiments, the dimensionality reduction comprises manifold learning.

In some embodiments, the manifold learning comprises uniform manifold approximation and projection (UMAP).

In some embodiments, the method further comprises determining, at least, a location-analyte estimation, for each location-specific-marker type of the plurality of different location-specific-marker types and for each analyte type of the plurality of different analyte types, of an estimated number of location-specific markers, of the plurality of location-specific markers and of the location-specific-marker type, associated with respective analytes of the analyte type.

Other aspects and features will become apparent to those ordinarily skilled in the art upon review of the following description of illustrative embodiments in conjunction with the accompanying figures.

### BRIEF DESCRIPTION OF THE DRAWINGS

FIG. 1 schematically illustrates an example of a spatial-sequencing method according to one embodiment.

FIG. 2 is an enlarged and fragmentary view of a bead of a monolayer sheet of FIG. 1.

FIG. 3 schematically illustrates an E. coli bacterium according to the embodiment of FIG. 1.

FIG. 4 schematically illustrates an example of producing multiple-location markers according to the embodiment of FIG. 1.

FIG. 5 illustrates an example of multiple-location markers producing according to the example of FIG. 4.

FIG. 6 illustrates examples of distributions of diameters of regions of multiple-location markers.

FIG. 7 schematically illustrates a paper disc of FIG. 5 stamped on a side the monolayer sheet of FIG. 1.

FIG. 8 schematically illustrates an example of further spatial sequencing according to one embodiment.

FIG. 9 schematically illustrates an example of a spatial-sequencing method according to another embodiment.

FIG. 10 schematically illustrates an example of a spatial-sequencing method according to another embodiment.

FIG. 11 schematically illustrates an example of a spatial-sequencing method according to another embodiment.

FIG. 12 and FIG. 13 schematically illustrate an example of a spatial-sequencing method according to another embodiment.

### DETAILED DESCRIPTION

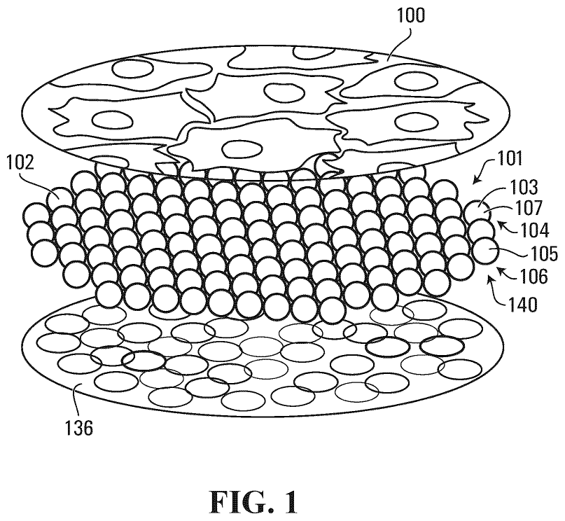

FIG. 1 schematically illustrates an example of a spatial-sequencing method according to one embodiment.

### Sample

The embodiment of FIG. 1 involves spatial sequencing of analytes in a sample 100. The sample 100 in the embodiment shown is cryogenically frozen animal tissue, but alternative embodiments may differ. For example, a sample of an alternative embodiment may include one or more biological samples such as one or more samples of animal tissue, one or more samples of plant material, one or more samples of fungal material, one or more samples of bacterial distribution, or a combination of two or more thereof. In some embodiments, a sample may be cryogenically frozen, paraffin-embedded, or both.

Also, the sample 100 in the embodiment shown is only several cells in diameter, but a sample of an alternative embodiment may be larger or smaller, and may for example be a tumor, a portion of an animal organ, an entire animal organ, a portion of an animal, or an entire animal.

### Analytes

A sample as described herein may include analytes. For example, in the embodiment shown, at least some of the analytes of the sample are messenger ribonucleic acid (mRNA) molecules, each having a respective nucleotide sequence. In general, such analytes may represent a transcriptome of at least one sample. However, alternative embodiments may differ, and analytes of alternative embodiments may include other molecules, other polymeric molecules, or other molecules each having a respective nucleotide sequence. For example, in some embodiments, such analytes may include ribonucleic acid (RNA) molecules, deoxyribonucleic acid (DNA) molecules, proteins, other analytes, or a combination of any two or more of the analytes described above. For example, in some embodiments, such analytes may include both RNA and DNA molecules. In some embodiments, some or all DNA molecules may have open chromatin regions as in assay for transposase-accessible chromatin with sequencing (ATAC-seq), for example.

Analytes of a sample as described herein may each be of respective analyte types. For example, mRNA molecules including nucleotides of one nucleotide sequence may be of one analyte type, and mRNA molecules including nucleotides of a different nucleotide sequence may be of a different analyte type. In other words, different RNA nucleotide sequences may be different analyte types. In general, an analyte as described herein may have a respective nucleotide sequence or may otherwise be of one analyte type and distinguishable from an analyte of a different analyte type.

As another example, in embodiments (as in ATAC-seq, for example) in which some or all of the analytes are DNA molecules having open chromatin regions, different nucleotide sequences of such open chromatin regions may be different analyte types. In other words, DNA molecules including open chromatin regions including nucleotides of one nucleotide sequence may be of one analyte type, and DNA molecules including open chromatin regions including nucleotides of a different nucleotide sequence may be of a different analyte type.

As another example, in embodiments in which some or all of the analytes are DNA molecules, different methylation patterns of DNA molecules may be different analyte types. In other words, DNA molecules having one methylation pattern may be of one analyte type, and DNA molecules having a different methylation pattern may be of a different analyte type.

As another example, in embodiments (as in Hi-C, for example) in which some or all of the analytes are DNA molecules, different DNA contact points may be different analyte types. In other words, DNA molecules having certain contact points may be of one analyte type, and DNA molecules having different contact points may be of a different analyte type.

As another example, in embodiments in which some or all of the analytes are proteins, different protein types may be different analyte types. For example, different protein types may be different amino-acid sequences, different structures of amino acids, or both. In other words, proteins having one sequence and structure of amino acids may be of one analyte type, and proteins having a different sequence or a different structure of amino acids may be of a different analyte type.

Embodiments may include other analyte types or combinations of different analyte types. For example, in some embodiments, analyte types may include different nucleotide sequences of open chromatin regions (as in ATAC-seq, for example) and different RNA nucleotide sequences.

### Beads or Other Bodies

The embodiment of FIG. 1 involves stamping the sample 100 on a first side (for example a top side) shown generally at 101 of a monolayer sheet 102 of sequencing beads such as reverse-transcription (RT) primer beads, for example. At least some of the beads may, for example, each be made of plastic and have a diameter of about 5 microns, of about 10 microns, or of about 30 microns. In some embodiments, the monolayer sheet 102 may be formed by spreading liquid silicone on a glass coverslip by centrifugation, and then spreading liquid rubber on the liquid silicone by further centrifugation, and then loading the sequencing beads on the liquid rubber with a plastic gasket and spreading the sequencing beads on the liquid rubber by further centrifugation such that the sequencing beads are half-embedded into the liquid rubber. However, alternative embodiments may differ.

In the embodiment of FIG. 1, each bead of the monolayer sheet 102 is at a respective different location. Such locations may be relative to the sample 100, relative to the monolayer sheet 102, or relative to another reference location, and each such location may have a respective two-dimensional (2D) or three-dimensional (3D) position in physical space relative to the sample 100, relative to the monolayer sheet 102, or relative to another reference location. For example, a bead 103 of the monolayer sheet 102 is at a location shown generally at 104 relative to the sample 100, relative to the monolayer sheet 102, or relative to another reference location, and a bead 105 of the monolayer sheet 102 and different from the bead 103 is at a location shown generally at 106 relative to the sample 100, relative to the monolayer sheet 102, or relative to another reference location and different from the location 104.

The beads of the monolayer sheet 102 are examples only, and alternative embodiments may differ. For example, an alternative embodiment may include other bodies, which may not necessarily be beads and may not necessarily be plastic. Also, in alternative embodiments, such beads or other bodies may not necessarily be in a monolayer sheet but rather may be distributed, held, or positioned at respective different locations in other ways, or such beads or other bodies may be omitted.

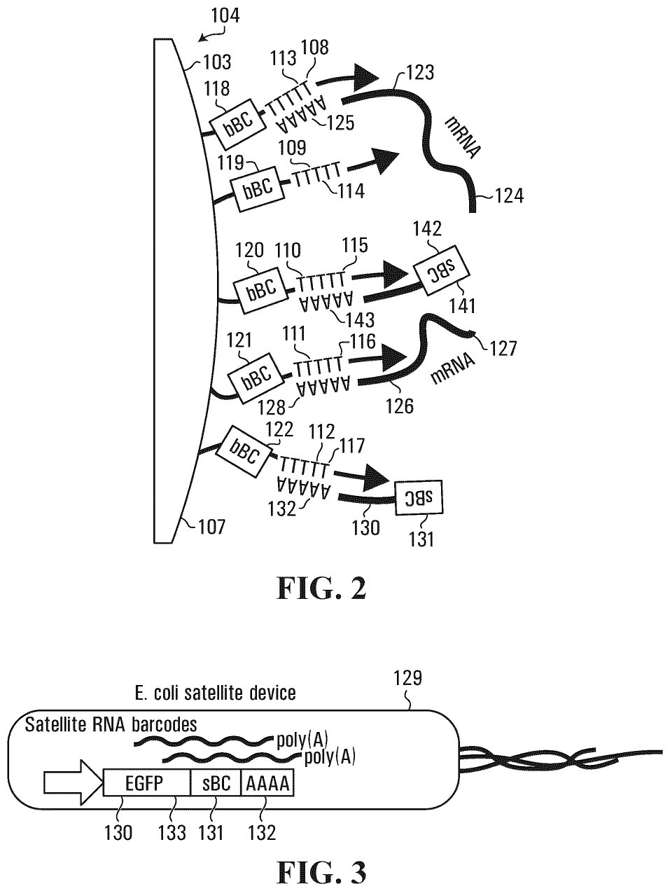

Referring to FIG. 2, the bead 103 has an outer surface 107, and RT primers (such as RT primers 108, 109, 110, 111, 112 as shown in FIG. 2) may be on the outer surface 107. At least some of the RT primers may include respective binding portions. For example, the RT primer 108 includes a binding portion 113, the RT primer 109 includes a binding portion 114, the RT primer 110 includes a binding portion 115, the RT primer 111 includes a binding portion 116, and the RT primer 112 includes a binding portion 117. In the embodiment shown, such a binding portion of an RT primer may include multiple thymine nucleotides, which may allow the RT primer to bind another molecule, for example by binding to a polyadenylated (poly(A)) tail of an mRNA molecule. More generally, some or all of the binding portions as described herein may be capable of binding to respective other molecules, such as RNA molecules for example.

However, alternative embodiments may differ. For example, an RT primer according to a different embodiment may include a different molecule, a different polymer, or a different molecule having a nucleotide sequence. Further, alternative embodiments may include one or more alternatives to RT primers.

### Location-Specific Markers

At least some of the RT primers may be encoded with respective location-specific markers. For example, the RT primer 108 is encoded with a location-specific marker 118, the RT primer 109 is encoded with a location-specific marker 119, the RT primer 110 is encoded with a location-specific marker 120, the RT primer 111 is encoded with a location-specific marker 121, and the RT primer 112 is encoded with a location-specific marker 122. In the embodiment shown, each such location-specific marker includes deoxyribonucleic acid (DNA) nucleotides having a respective nucleotide sequence.

Location-specific markers as described herein may each be of a respective location-specific-marker type. For example, a location-specific marker including nucleotides of one nucleotide sequence may be of one location-specific-marker type, and a location-specific marker including nucleotides of a different nucleotide sequence may be of a different location-specific-marker type.

Location-specific markers according to other embodiments may differ. However, in general, a location-specific marker as described herein may have a respective nucleotide sequence or may otherwise be of one location-specific-marker type and distinguishable from a location-specific marker of a different location-specific-marker type.

In general, some or all of a plurality of location-specific markers may be at (and therefore associated with) respective different locations (for example, the locations 104 and 106) of a plurality of different locations (for example, respective locations of some or all of the beads of the monolayer sheet 102). For example, the location-specific markers 118, 119, 120, 121, and 122 are all at (and therefore associated with) the location 104 of the bead 103. Other location-specific markers may be at (and therefore associated with) the location 104 of the bead 103, and still other location-specific markers may be at (and therefore associated with) respective other locations associated with respective other beads of the monolayer sheet 102.

In some embodiments, some or all of the location-specific markers at (or associated with) one location (such as the location 104 of the bead 103, for example) may be of one location-specific-marker type, and that location-specific-marker type may be associated with that location. For example, some or all of the location-specific markers 118, 119, 120, 121, and 122 at (or associated with) the location 104 of the bead 103 may include a nucleotide sequence (or otherwise be of a location-specific-marker type) associated with the location 104.

Further, in some embodiments, some or all of the location-specific markers at (or associated with) a different location (such as the location 106 of the bead 105, for example) may be of a different location-specific-marker type, and that different location-specific-marker type may be associated with that other location. For example, some or all of the location-specific markers at (or associated with) the location 106 of the bead 105 may include nucleotides of a nucleotide sequence (or otherwise be of a location-specific-marker type) associated with the location 106 and different from the nucleotide sequence (or location-specific-marker type) associated with the location 104.

In general, a plurality of locations (such as respective locations associated with some or all of the beads of the monolayer sheet 102) may each be associated with a respective different location-specific-marker type.

Also, in some embodiments, location-specific markers as described herein may be associated with respective beads and may therefore be referred to as bead-specific barcodes (bBCs). However, as indicated above, alternative embodiments may include other bodies, which may not necessarily be beads, or such beads or other bodies may be omitted.

Analytes Becoming Associated with Respective Location-Specific Markers

As indicated above, a binding portion of an RT primer may include multiple thymine nucleotides, which may allow the RT primer to bind another molecule, for example by binding to a poly(A) tail of an mRNA molecule. For example, FIG. 2 illustrates an analyte 123 including an mRNA portion 124 and a poly(A) tail 125 bound to the binding portion 113 of the RT primer 108, and an analyte 126 including an mRNA portion 127 and a poly(A) tail 128 bound to the binding portion 116 of the RT primer 111. As a result, the analyte 123 is bound to (or, more generally, becomes associated with) the location-specific marker 118 of the RT primer 108, and the analyte 126 is bound to (or, more generally, becomes associated with) the location-specific marker 121 of the RT primer 111. Herein, reference to binding does not necessarily refer to binding directly, and may refer to binding indirectly. For example, in the embodiment of FIG. 2, the analyte 123 is bound to the location-specific marker 118 without necessarily being bound directly, and the analyte 126 is bound to the location-specific marker 121 without necessarily being bound directly.

In other words, in the embodiment of FIG. 1, stamping the sample 100 on the first side 101 of the monolayer sheet 102 may cause a plurality of analytes (for example, the analytes 123 and 126) from at least one sample (for example, the sample 100) to bind to (or, more generally, to become associated with), at least, respective location-specific markers (for example, the location-specific markers 118 and 121, respectively) of a plurality of location-specific markers (including other location-specific markers of the bead 103 and location-specific markers of other beads of the monolayer sheet 102).

Of course the bead 103, the RT primers 108 and 111, and the analytes 123 and 126 are examples only, and stamping the sample 100 on the first side 101 of the monolayer sheet 102 may cause many different analytes to bind to (or, more generally, to become associated with) many respective different location-specific markers of many different beads of the monolayer sheet 102.

### Multiple-Location Markers

Referring to FIG. 3, an Escherichia coli (E. coli) bacterium 129 may create multiple-location molecules such as a multiple-location molecule 130. The multiple-location molecule 130 includes a DNA portion 131, a poly(A) tail 132, and a fluorescent protein 133 (enhanced green fluorescent protein (EGFP) in the embodiment shown). A DNA portion such as the DNA portion 131 may be referred to as a multiple-location marker in some embodiments, and the E. coli bacterium 129 may be referred to as a source of multiple-location molecules produced by the E. coli bacterium 129, and as a source of the multiple-location markers of the multiple-location molecules produced by the E. coli bacterium 129.

In general, the multiple-location marker (such as the DNA portion 131, for example) of a multiple-location molecule as described herein may be of a multiple-location-marker type. For example, a DNA portion (or multiple-location marker) of one multiple-location molecule and including nucleotides of one nucleotide sequence may be of one multiple-location-marker type, and a DNA portion (or multiple-location marker) of a different multiple-location molecule and including nucleotides of a different nucleotide sequence may be of a different multiple-location-marker type. In general, a multiple-location marker as described herein may have a respective nucleotide sequence or may otherwise be of one multiple-location-marker type and distinguishable from a multiple-location marker of a different multiple-location-marker type.

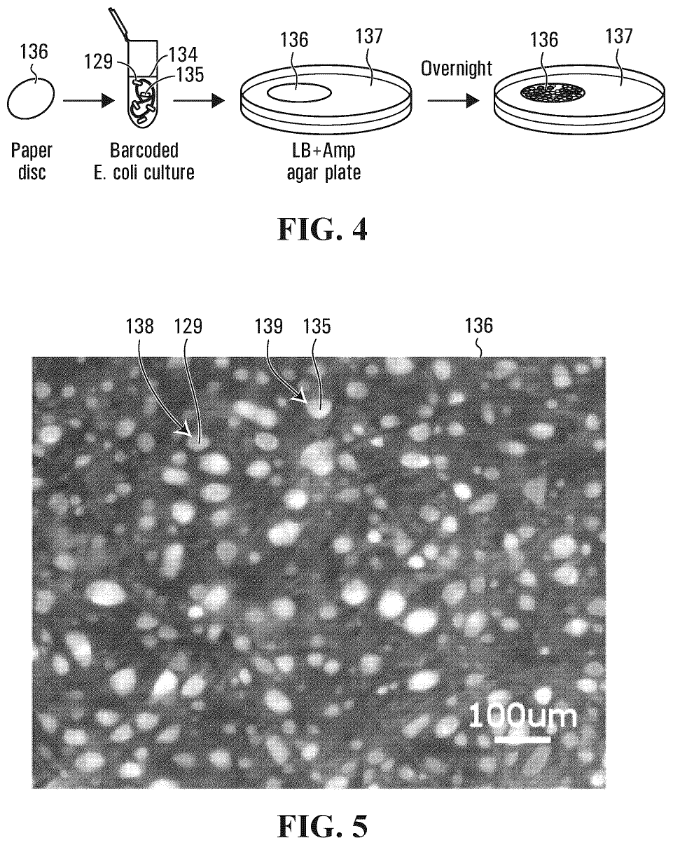

Referring to FIG. 4, an E. coli bacterial culture 134 may be formed with many different E. coli bacteria similar to the E. coli bacterium 129. For example, in the embodiment shown, the E. coli bacterial culture 134 includes the E. coli bacterium 129 and a different E. coli bacterium 135.

In some embodiments, some or all of the multiple-location molecules produced by one E. coli bacterium of the E. coli bacterial culture 134 may include nucleotides of the same nucleotide sequence and may therefore be of the same multiple-location-marker type. For example, in some embodiments, some or all of the multiple-location molecules produced by the E. coli bacterium 129 may include nucleotides of one nucleotide sequence and may therefore be of one multiple-location-marker type.

Further, in some embodiments, some or all of the multiple-location molecules produced by a different E. coli bacterium of the E. coli bacterial culture 134 may include respective multiple-location markers of a different multiple-location-marker type. For example, some or all of the multiple-location molecules produced by the E. coli bacterium 135 may include respective multiple-location markers including nucleotides of a nucleotide sequence (or otherwise be of a multiple-location-marker type) different from the nucleotide sequence (or multiple-location-marker type) of multiple-location molecules produced by the E. coli bacterium 129. The E. coli bacterium 135 may be referred to as a source of multiple-location molecules produced by the E. coli bacterium 135, and as a source of the multiple-location markers of the multiple-location molecules produced by the E. coli bacterium 135.

More generally, an E. coli bacterium of the E. coli bacterial culture 134 may be referred to as a source of multiple-location molecules produced by the E. coli bacterium, and as a source of the multiple-location markers of the multiple-location molecules produced by the E. coli bacterium.

A paper disc 136 (such as a chromatography paper, for example) may be soaked in the E. coli bacterial culture 134 such that the E. coli bacteria of the E. coli bacterial culture 134 are distributed in or on the paper disc 136. The paper disc 136 may then be placed on a solid Luria broth (LB) agar plate 137, allowing the E. coli bacteria of the E. coli bacterial culture 134 to produce multiple-location molecules in or on the paper disc 136, thereby causing multiple-location molecules to propagate from the E. coli bacteria producing the multiple-location molecules and causing multiple-location markers of the multiple-location molecules to propagate from sources of the multiple-location markers. In some embodiments, the paper disc 136 may be wet with LB and ampicillin and placed on a dish with LB, ampicillin, and agar, and a 2% solution of LB and agarose mixed with E. coli cells may be spread over the paper disc 136. A glass coverslip may then be placed on the liquid gel to make it flat and thin, and the coverslip may be pealed off after the liquid gel solidifies. In other embodiments, instead of mixing liquid LB, agarose, and E. coli cell culture media, the culture media may be spread on a thin gel. In some embodiments, the paper disc 136 may be frozen at −80° C. and gently soaked in a lysis buffer.

Propagation of multiple-location markers may involve free diffusion or electrophoresis blotting, for example. In some embodiments, such electrophoresis blotting may involve electrophoretically blotting gels onto a positively charged nylon transfer membrane using a Southern blotting apparatus and staining for DNA on the membrane using a methylene blue dye mixture. In some embodiments, 20 minutes of alkaline denaturation and 14 minutes of blotting may show the strongest SYBR™ fluorescence reduction and transfer membrane staining, respectively.

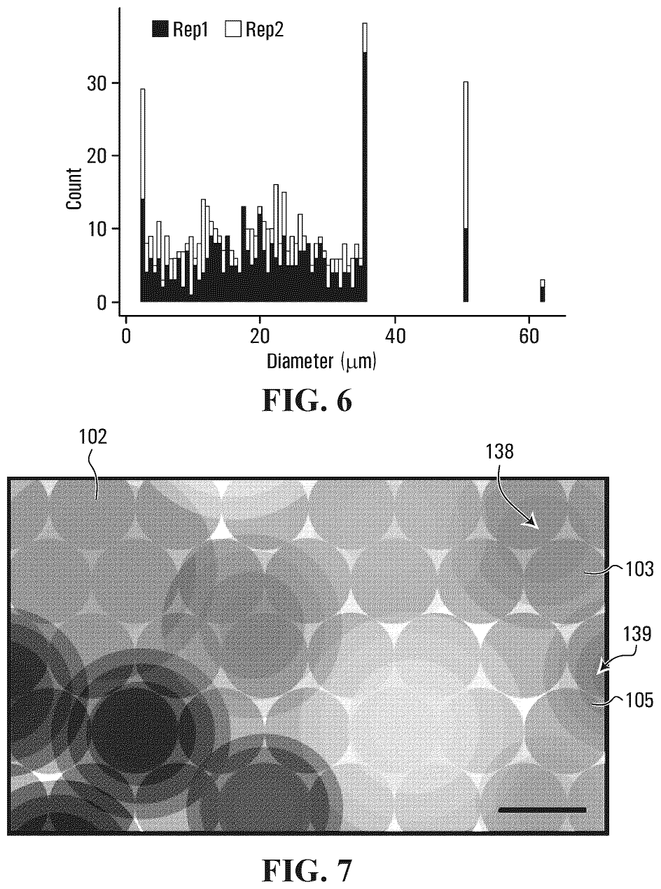

In general, some or all of the E. coli bacteria of the E. coli bacterial culture 134 may produce multiple-location molecules in respective regions in or on the paper disc 136. For example, as shown in FIG. 5, the E. coli bacterium 129 may produce multiple-location molecules (each including a respective multiple-location marker such as the DNA portion 131 of one multiple-location-marker type) in a region shown generally at 138, and the E. coli bacterium 135 may produce multiple-location molecules (each including a respective multiple-location marker such as the DNA portion 131 of one multiple-location-marker type but of a different multiple-location-marker type from multiple-location markers of multiple-location molecules produced by the E. coli bacterium 129) in a region shown generally at 139. The region 138 is associated with the multiple-location-marker type of multiple-location molecules produced by the E. coli bacterium 129, and the region 139 is associated with the multiple-location-marker type of multiple-location molecules produced by the E. coli bacterium 135. FIG. 6 illustrates a distribution of diameters of such regions according to one embodiment (identified in FIG. 6 as Rep1) and according to another embodiment (identified in FIG. 6 as Rep2). Other embodiments may differ.

In general, when multiple-location molecules propagate from a source of the multiple-location molecules, the multiple-location molecules (and the multiple-location markers of the multiple-location molecules) may be in concentrations or abundances that are positively correlated with relative proximities of the multiple-location molecules to the source of the multiple-location molecules (or, more generally, to reference locations of the multiple-location-marker types). Therefore, multiple-location markers of a multiple-location-marker type may be at locations in or on the paper disc 136 in concentrations or abundances that are positively correlated with relative proximities of the multiple-location markers to a source of the multiple-location markers of the multiple-location-marker type (or, more generally, to a reference location of the multiple-location-marker type).

The E. coli bacteria of the E. coli bacterial culture 134 are an example only, and alternative embodiments may differ. For example, in other embodiments, sources of multiple-location-markers may include other bacteria, polymerases, other enzymes, one or more other alternatives to the E. coli bacteria of the E. coli bacterial culture 134, or a combination of two or more thereof.

### Multiple-Location Markers Becoming Associated with Respective Location-Specific Markers

The embodiment of FIG. 1 also involves exposing the beads of the monolayer sheet 102 to some or all of the multiple-location markers in or on the paper disc 136 by stamping the paper disc 136 on a second side (for example a bottom side) shown generally at 140 and opposite the first side 101 of the monolayer sheet 102. In the embodiment of FIG. 1, stamping the paper disc 136 on the second side 140 of the monolayer sheet 102 causes some or all of the multiple-location markers in or on the paper disc 136 to be transferred from at least one surface (in the embodiment of FIG. 1, a surface of the paper disc 136) to location-specific markers on beads of the monolayer sheet 102.

FIG. 7 illustrates schematically the paper disc 136 stamped on the second side 140 of the monolayer sheet 102. As shown in FIG. 7, the multiple-location markers in the region 138 are exposed to multiple beads of the monolayer sheet 102, including the bead 103, and the multiple-location markers in the region 139 are exposed to multiple beads of the monolayer sheet 102, including the beads 103 and 105. More generally, multiple-location markers in a region (for example, the region 138 or 139) may bind to (or, more generally, to become associated with), at least, respective location-specific markers at multiple locations (for example, the locations 104 and 106 or other locations associated with respective beads of the monolayer sheet 102), and the locations, of location-specific markers that multiple-location markers of a multiple-location-marker type become associated with, may be associated with the multiple-location-marker type.

As indicated above, multiple-location markers of a multiple-location-marker type may be at locations in or on the paper disc 136 in concentrations or abundances that are positively correlated with relative proximities of the multiple-location markers to a source of the multiple-location markers of the multiple-location-marker type (or, more generally, to a reference location of the multiple-location-marker type). Therefore, multiple-location markers of a multiple-location-marker type may bind to (or, more generally, to become associated with), at least, respective location-specific markers at multiple locations in concentrations or abundances that are positively correlated with relative proximities of the locations to a source of the multiple-location markers of the multiple-location-marker type (or, more generally, to a reference location of the multiple-location-marker type).

For at least those reasons, multiple-location markers as described herein may be analogous to signals from respective satellites in a global positioning system (GPS), and a DNA portion such as the DNA portion 131 may be referred to as a satellite barcode (sBC) in some embodiments. However, any analogy to a GPS or to any satellites is for illustration only and not intended to limit the scope of the disclosure.

In FIG. 2, the poly(A) tail 132 of the multiple-location molecule 130 is bound to the binding portion 117 of the RT primer 112, and the DNA portion 131 is bound to (or, more generally, becomes associated with) the location-specific marker 122 of the RT primer 112. Further, a multiple-location molecule 141, similar to the multiple-location molecule 130 but produced by the E. coli bacterium 135, has a DNA portion 142 and a poly(A) tail 143 bound to the binding portion 115 of the RT primer 110, and the DNA portion 142 binds to (or, more generally, becomes associated with) the location-specific marker 120 of the RT primer 110. Because the multiple-location molecule 130 was produced by the E. coli bacterium 129 and the multiple-location molecule 141 was produced by the E. coli bacterium 135, the DNA portions 130 and 141 have different nucleotide sequences and are of different multiple-location-marker types.

Of course the bead 103, the RT primers 110 and 112, and the multiple-location molecules 130 and 141 are examples only, and exposing the beads of the monolayer sheet 102 to the multiple-location markers on the in or on the paper disc 136 by the paper disc 136 on the second side 140 of the monolayer sheet 102 may cause many different multiple-location markers to bind to (or, more generally, to become associated with) many respective different location-specific markers of many different beads of the monolayer sheet 102.

In other words, in the embodiment of FIG. 1, exposing the beads of the monolayer sheet 102 to the multiple-location markers in or on the paper disc 136 may cause a plurality of multiple-location markers (for example, the DNA portions 130 and 141) to bind to (or, more generally, to become associated with), at least, respective location-specific markers (for example, the location-specific markers 122 and 120, respectively) of a plurality of location-specific markers (including other location-specific markers of the bead 103 and location-specific markers of other beads of the monolayer sheet 102).

### Location-Analyte Estimations

In the embodiment of FIG. 1, when an analyte binds to a location-specific marker, as shown in FIG. 2 for example, a complementary DNA (cDNA) molecule may be produced, using reverse transcriptase, from the molecule that results from the analyte binding to the location-specific marker, and the cDNA molecule may be replicated (or amplified) by polymerase chain reaction (PCR). The cDNA molecule, and the PCR replicates of the cDNA molecule, represent the analyte (or a portion of the analyte) and the location-specific marker by including a nucleotide sequence complementary to the analyte (or to a portion of the analyte) and a nucleotide sequence complementary to the location-specific marker.

For example, after the analyte 123 is bound to the location-specific marker 118, a cDNA molecule may be produced, using reverse transcriptase, from the resulting molecule (the resulting molecule including the location-specific marker 118 and the mRNA portion 124), and the cDNA molecule may be replicated by PCR. The cDNA molecule, and the PCR replicates of the cDNA molecule, represent the analyte 123 (or the mRNA portion 124 of the analyte 123) and the location-specific marker 118 by including a nucleotide sequence complementary to the analyte 123 (or to the mRNA portion 124 of the analyte 123) and a nucleotide sequence complementary to the location-specific marker 118.

In general, each molecule that results from an analyte binding to a location-specific marker indicates the analyte at the location of the location-specific marker. Therefore, a cDNA molecule produced, using reverse transcriptase, from a molecule that results from an analyte binding to a location-specific marker, and the PCR replicates of the cDNA molecule, also indicate the analyte at the location of the location-specific marker.

Deep sequencing may be used to estimate numbers of such cDNA molecules, and PCR replicates of such cDNA molecules, for different pairs of location-specific-marker types and analyte types. Such an estimated number, of such cDNA molecules and PCR replicates of such cDNA molecules, for a location-specific-marker type and an analyte type may be proportional to a number of analytes of the analyte type at a location associated with the location-specific-marker type. Such an estimated number of analytes of an analyte type, at a location associated with a location-specific-marker type, may be referred to as a location-analyte estimation for that location-specific-marker type and that analyte type. Deep sequencing is an example only, and alternative embodiments may involve estimating in other ways.

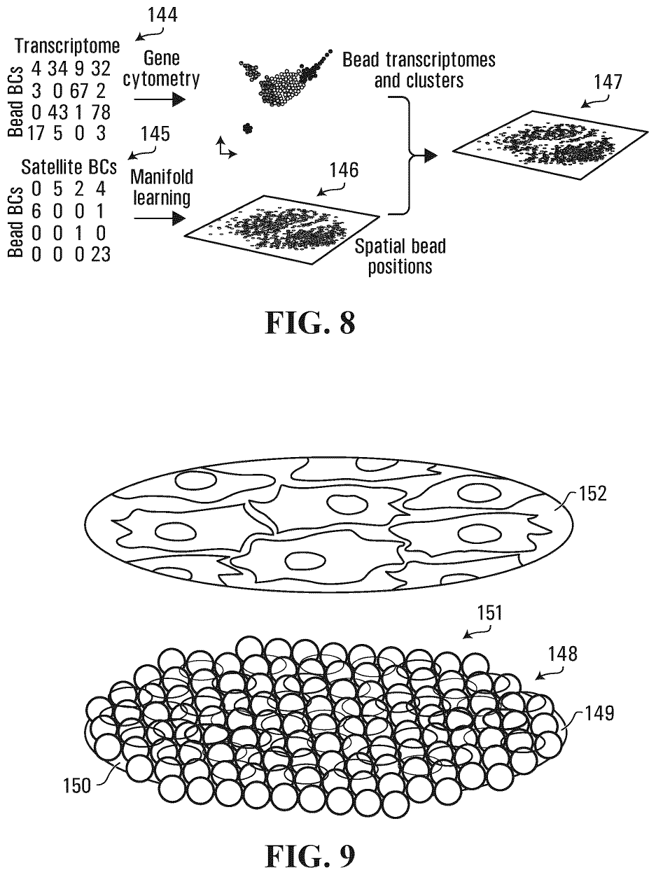

Referring to FIG. 8, a location-analyte matrix shown generally at 144 may include such location-analyte estimations for pairs of different location-specific-marker types and different analyte types. For example, in the location-analyte matrix 144, each row represents one location-specific-marker type, and each column represents one analyte type. A value at a column and at a row in the location-analyte matrix 144 is an estimate of a number of analytes, of an analyte type of that column, at a location associated with a location-specific-marker type of that row. The location-analyte matrix 144 is only an example illustration, and alternative embodiments are not necessarily limited to any specific representations or data structures.

### Multiple-Location-Marker Estimations

Likewise, in the embodiment of FIG. 1, when a multiple-location marker binds to a location-specific marker, as shown in FIG. 2 for example, a cDNA molecule may be produced, using reverse transcriptase, from the molecule that results from the multiple-location marker binding to the location-specific marker, and the cDNA molecule may be replicated (or amplified) by PCR. The cDNA molecule, and the PCR replicates of the cDNA molecule, represent the multiple-location marker and the location-specific marker by including a nucleotide sequence complementary to the multiple-location marker and a nucleotide sequence complementary to the location-specific marker.

For example, after the DNA portion 131 (a multiple-location marker) is bound to the location-specific marker 122, a cDNA molecule may be produced, using reverse transcriptase, from the resulting molecule (the resulting molecule including the location-specific marker 122 and the DNA portion 131), and the cDNA molecule may be replicated by PCR. The cDNA molecule, and the PCR replicates of the cDNA molecule, represent the DNA portion 131 and the location-specific marker 122 by including a nucleotide sequence complementary to the DNA portion 131 and a nucleotide sequence complementary to the location-specific marker 122.

In general, each molecule that results from a multiple-location marker binding to a location-specific marker indicates the multiple-location marker at the location of the location-specific marker. Therefore, a cDNA molecule produced, using reverse transcriptase, from a molecule that results from multiple-location marker binding to a location-specific marker, and the PCR replicates of the cDNA molecule, also indicate the multiple-location marker at the location of the location-specific marker.

Deep sequencing may be used estimate numbers of such cDNA molecules, and PCR replicates of such cDNA molecules, for different pairs of location-specific-marker types and multiple-location-marker types. Such an estimated number, of such cDNA molecules and PCR replicates of such cDNA molecules, for a location-specific-marker type and a multiple-location-marker type may be proportional to a number of multiple-location markers of the multiple-location marker type at a location associated with the location-specific-marker type. Such an estimated number of multiple-location markers of a multiple-location-marker type, at a location associated with a location-specific-marker type, may be referred to as a multiple-location-marker estimation for that location-specific-marker type and that multiple-location-marker type. Deep sequencing is an example only, and alternative embodiments may involve estimating in other ways.

Referring to FIG. 8, a multiple-location-marker matrix shown generally at 145 may include such multiple-location-marker estimations for pairs of different location-specific-marker types and multiple-location-marker types. For example, in the multiple-location-marker matrix 145, each row represents one location-specific-marker type, and each column represents one multiple-location-marker type. A value at a column and at a row in the multiple-location-marker matrix 145 is an estimate of a number of multiple-location markers, of a multiple-location-marker type of that column, at a location associated with a location-specific-marker type of that row. The multiple-location-marker matrix 145 is only an example illustration, and alternative embodiments are not necessarily limited to any specific representations or data structures.

### Dimensionality Reduction

The location-analyte matrix 144 and the multiple-location-marker matrix 145 are 4×4 matrices for simplicity of illustration, but similar matrices in other embodiments may be significantly larger.

The multiple-location-marker matrix 145 has a row for each location and a column for each multiple-location marker. A value in the multiple-location-marker matrix 145 in a row i and in a column j indicates an abundance of multiple-location markers of a multiple-location-marker type associated with the column j measured at a location associated with the row i. Therefore, the location associated with the row i is associated with a large list, or vector, of multiple-location marker abundances (namely the values in the multiple-location-marker matrix 145 in the row i). The position, in physical 2D or 3D space, of a location associated with a location-specific-marker type can be recovered from such a list of abundances.

Dimensionality reduction is one way to recover the respective positions, in physical 2D or 3D space, of locations associated with respective location-specific-marker types. The vector of multiple-location marker abundances associated a location-specific-marker type (corresponding to a row of the multiple-location-marker matrix 145) is a representation of the position, in physical 2D or 3D space, of a location associated with the location-specific-marker type in a high-dimensional space of multiple-location marker abundances. The process of identifying multiple-location-marker estimations, as described above for example, can be understood to embed the respective positions, in physical 2D or 3D space, of locations associated with respective location-specific-marker types into this high-dimensional space of multiple-location marker abundances (with a dimension for each multiple-location-marker type). According to a mathematical theorem, the respective positions, in physical 2D or 3D space, of locations associated with respective location-specific-marker types form respective low-dimensional manifolds continuously embedded in this high-dimensional space. Such a low-dimensional manifold is of a dimension equal to the dimension of the original physical space. For example, when respective positions of locations associated with respective location-specific-marker types are on at least one surface of at least one sample, such low-dimensional manifolds may be 2D, and when respective positions of locations associated with respective location-specific-marker types are in at least one sample, such low-dimensional manifolds may be 3D. In general, dimensionality reduction, or “manifold learning” techniques, can be applied to recover the respective positions of respective locations associated with some or all of the location-specific-marker types, as shown generally at positions 146 in FIG. 8. This dimensionality reduction could be performed via uniform manifold approximation and projection (UMAP). Regarding UMAP, McInnes et al., “UMAP: Uniform Manifold Approximation and Projection for Dimension Reduction”, cited as arXiv:1802.03426, may assist the reader.

In some embodiments, one or more processor circuits may include one or more processors (such as one or more central processing unit (CPUs) or microprocessors, one or more machine learning chips, one or more discrete logic circuits, or one or more application-specific integrated circuit (ASICs), or combinations of two or more thereof). Such one or more processor circuits may also include one or more of the same or different computer-readable storage media, which in various embodiments may include one or more read-only memory (ROM), one or more random access memory (RAM), one or more hard disc drive (HDD), one or more solid-state drive (SSD), or one or more other computer-readable and/or computer-writable storage media, or combinations of two or more thereof. Such one or more such computer-readable storage media may store program codes that, when executed by the one or more processors, cause the one or more processors to, at least,
1. receive (at one or more signal interfaces of the one or more processor circuits, for example) one or more signals identifying multiple-location-marker estimations, identified by deep sequencing as described above for example, and
2. identify respective positions, in physical 2D or 3D space, of respective locations associated with some or all of the location-specific-marker types, for example using dimensionality reduction or manifold learning as described above,
   or such one or more processor circuits may be programmed, configured, or operable to implement at least such functions in other ways.

### Identifying Analytes at Positions

Once respective positions, in physical 2D or 3D space, of respective locations associated with some or all of the location-specific-marker types are identified, as described above, for example, respective abundances of analytes of different analyte types at such locations may be identified. For example, once a position, in physical 2D or 3D space, is identified of a location associated with a location-specific-marker type or with a row i of the multiple-location-marker matrix 145, then the same row i of the location-analyte matrix 144 indicates estimates of numbers of analytes, of different analyte types, at that position.

One or more processor circuits, as described above for example, may also be programmed, configured, or operable to, at least, identify respective abundances of analytes of different analyte types at respective positions, in physical 2D or 3D space, of respective locations associated with some or all of the location-specific-marker types, which can then be used to form an image (for example, as shown generally at 147 in FIG. 8) by illustrating respective abundances of analytes of different analyte types from the location-analyte matrix 144 at different positions as identified above, for example. One or more processor circuits, as described above for example, may also be programmed, configured, or operable to, at least, produce one or more output signals (at one or more signal interfaces of the one or more processor circuits, for example) identifying at least such an image or such images.

### Examples of Mathematical Models

According to one possible mathematical model, X is a compact subset of [symbol] ² and defines a domain of interest (for example, [0, 1]² or a unit disk). A source of multiple-location markers j is a compact connected set Sⱼ⊂X, which can be a small region of the domain, or a single point. A concentration or abundance of multiple-location markers from the source of multiple-location markers j (or of multiple-location markers of a multiple-location marker type j) may be defined as ψⱼ: X→ [symbol] ₊, which may be a continuous decreasing function of distance from the source of multiple-location markers j. To ensure that the multiple-location markers propagate collectively throughout the domain X, the sources of multiple-location markers would have to be distributed such that, throughout X, ψⱼ>0 for at least one j.

For a total of M sources of multiple-location markers (and therefore M multiple-location-marker types), a vector ψ of concentrations or abundances may be defined such that

$$ψ : X ↦ {ℝ}_{+}^{M}$$

$$x ↦ {[ {ψ}_{1} ( x ) , {ψ}_{2} ( x ) , … , {ψ}_{M} ( x ) ]}^{T}$$

and ψ represents concentrations or abundances of multiple-location markers at locations in X, and [symbol] ₊^{M} is a high-dimensional space of multiple-location marker abundances.

N sequencing beads or other bodies may have respective locations x₁, x₂, . . . , x_{N} embedded in a high-dimensional space ψ(x₁), ψ(x₂), . . . , ψ(x_{N}), and a two-dimensional surface of such beads or other bodies (or, more generally, of locations of N location-specific-marker types of location-specific markers) may form a manifold [symbol] in that space. Mathematically, [symbol] may be the image of X under ψ.

A probability of multiple-location markers of a multiple-location marker type j associated with a location i (or with a location-specific-marker type i) may be defined as

$${p}_{i j} = \frac{{ψ}_{j} ( {x}_{i} )}{{\sum }_{i , j} {q}_{i} {ψ}_{j} ( {x}_{i} )}$$

where qᵢ represents a quality of a sequencing bead (or other body) i. In a simple model, qᵢ=1. However, in some other models, Q may be a distribution on (0, 1], which may be generated from a distribution of counts of location-specific markers, and each qᵢ may be assigned as qᵢ˜Q.

A sequencing process of R samples may produce an N×M matrix C of counts including integer numbers of counts of multiple-location markers of each of the M multiple-location-marker types associated with each of the N location-specific-marker types. For example, C may be C˜Multi(R, [p₁₁, . . . , p_{NM}]), which may be a sparse matrix.

In practice, some multiple-location markers from different sources of the multiple-location markers may have the same multiple-location-marker type, which may be reflected in a model with a matrix Ĉ=PC where P is a projection operator.

Sources of multiple-location markers may have respective locations yⱼ∈ [symbol] ² such that ψⱼ(x)=g (∥x−yⱼ∥) where g: R₊→R₊ is a decreasing continuous bijection representing radial decay of concentration or abundance. In a Gaussian diffusion model,

$$g : x ↦ {e}^{\frac{- {x}^{2}}{{σ}^{2}}} ,$$

where σ is a Gaussian width that may vary for different diffusion levels.

If M≥3 and j₁, j₂, j₃∈[1, M] exist such that yⱼ₁, yⱼ₂, yⱼ₃ are non-colinear, then ψ is a smooth embedding of X⊂ [symbol] ² into [symbol] =ψ(X)∈ [symbol] ₊^{M}, and [symbol] is a smooth sub-manifold of [symbol] ₊^{M} as the image of ψ. Then, UMAP may be used to identify [symbol] , which may identify the locations x₁, x₂, . . . , x_{N} in two-dimensional space.

Once the locations x₁, x₂, . . . , x_{N} are identified in two-dimensional space, a location-analyte matrix (such as the location-analyte matrix 144) may identify analytes, and respective quantities of analytes of one or more analyte types, at those locations.

The model described above involves locations in two-dimensional space, but alternatives may involve locations in two-dimensional space.

### Computer Simulations

Computer simulations may be used to simulate accuracy according to models as described above, and such models may facilitate identification accuracy for different sequencing parameters.

For example, some computer simulations suggest that a median reconstruction distance may be below 20 microns using 100,000 sources of multiple-location molecules per square centimeter (cm²), σ of 50 microns, 130 unique molecular identifiers (UMIs) per sequencing bead, and 556 sequencing reads per sequencing bead.

Other computer simulations suggest that a median reconstruction distance may be below 10 microns using 180 UMIs per sequencing bead and 1,100 sequencing reads per sequencing bead.

Other computer simulations suggest that a median reconstruction distance may be below 30 microns using 15 UMIs per sequencing bead and 30 sequencing reads per sequencing bead.

Other computer simulations suggest that a median reconstruction distance may be below 20 microns in most cases using between 25,000 and 250,000 sources of multiple-location molecules per cm², σ of between 30 microns and 100 microns, and up to 265 UMIs per sequencing bead.

For example, some computer simulations suggest that a median reconstruction distance may be 10 microns using between 100,000 and 250,000 sources of multiple-location molecules per cm² and σ of between 30 microns and 50 microns, and other computer simulations suggest that a median reconstruction distance may be 30 microns using between 25,000 and 250,000 sources of multiple-location molecules per cm² and σ of between 30 microns and 100 microns.

Some computer simulations suggest that the largest reconstruction distances were at the smallest amount of diffusion, suggesting that adequate diffusion may be important.

In some embodiments, the optimal a may be about 50 microns for ten-micron beads.

However, alternative embodiments may differ.

### Alternative Involving Beads on a Slide

FIG. 9 schematically illustrates an example of a spatial-sequencing method according to another embodiment. In the embodiment of FIG. 9, a layer shown generally at 148 of beads (or other bodies) as described above, for example, may be similar to the monolayer sheet 102 as described above, but may be adhered to a surface (in the embodiment shown, a surface 149 of a glass microscope slide 150, although surfaces of alternative embodiments may differ). Multiple-location molecules (similar to multiple-location molecules as described above, for example), each including a respective multiple-location marker, may be transferred (for example, from a surface of a paper disc that may be similar to the paper disc 136 as described above) to the beads of the layer 148 on a side shown generally at 151 of the beads opposite the glass microscope slide 150. Further, a sample 152 may be similar to the sample 100 and may also be stamped on the beads of the layer 148 on the side 151. The embodiment of FIG. 9 may otherwise be similar to the embodiments described above.

### Alternative Involving Amplifying Multiple-Location Markers in a Gel

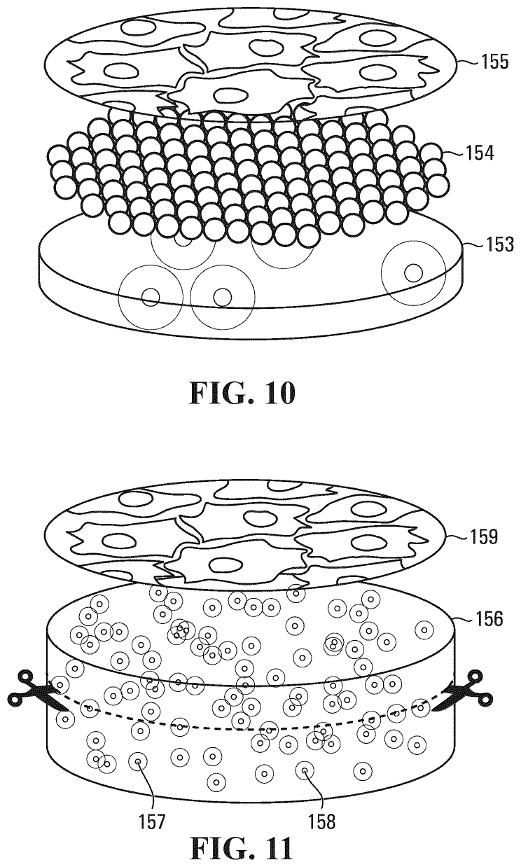

FIG. 10 schematically illustrates an example of a spatial-sequencing method according to another embodiment. In the embodiment of FIG. 10, multiple-location molecules (similar to multiple-location molecules as described above, for example), each including a respective multiple-location marker, may be created in a gel 153. In different embodiments, a gel as described herein may include polyacrylamide gel, a polyethylene glycol gel, one or more other gels, or two or more thereof, for example.

In some embodiments, different DNA molecules may be distributed within the gel 153, and each such DNA molecule may be a source of multiple-location molecules, each including a respective multiple-location marker of a respective multiple-location-marker type. Such DNA molecules may be amplified such that multiple-location molecules including respective multiple-location markers of different multiple-location-marker types propagate in the gel 153 from respective sources of multiple-location molecules. The multiple-location markers of multiple-location molecules may be of a multiple-location-marker type associated with the source of the multiple-location molecules, and the multiple-location-marker types associated with some or all of the source of multiple-location molecules may differ from the multiple-location-marker types associated with some or all of the other sources of multiple-location molecules. In other words, the multiple-location molecules in such an embodiment may be created as polonies.

Then, as shown in FIG. 10, the gel 153 may be stamped on one side of a monolayer sheet 154 (similar to the monolayer sheet 102, for example), a sample 155 (similar to the sample 100 or 152, for example) may be stamped on the other side of the monolayer sheet 154, and the embodiment of FIG. 10 may otherwise be similar to the embodiments described above.

In the embodiments described above, a plurality of analytes become associated with respective location-specific markers, and a plurality of multiple-location markers become associated with respective location-specific markers, when the plurality of location-specific markers are bound to bodies such as beads.

Also, in the embodiment of FIG. 10, the plurality of multiple-location markers become associated with the respective location-specific markers when the plurality of multiple-specific markers are suspended in a gel.

However, alternative embodiments may differ, as described below for example.

### Alternative Involving Amplifying Location-Specific Markers in a Gel

FIG. 11 schematically illustrates an example of a spatial-sequencing method according to another embodiment. In the embodiment of FIG. 11, multiple-location molecules (similar to multiple-location molecules as described above, for example) may be created in a gel 156 as described above in the embodiment of FIG. 10, for example. However, in the embodiment of FIG. 11, location-specific molecules (shown at 157 and 158, for example) may also be created in the gel 156 by distributing, within the gel 156, different DNA molecules, each a source of location-specific molecules including respective location-specific markers of a location-specific-marker type associated with the source of location-specific molecules, and the location-specific-marker types associated with some or all of the sources of location-specific molecules may differ from the location-specific-marker types associated with some or all of the other sources of location-specific molecules.

Such DNA molecules may be amplified such that location-specific molecules including respective location-specific markers of different location-specific-marker types propagate in the gel 156 from respective such sources. The location-specific molecules may propagate from their respective sources within regions that are relatively small compared to regions in which multiple-location molecules propagate from their respective sources, for example by allowing propagation of multiple-location molecules during a greater period of time. In other words, the multiple-location molecules and the location-specific molecules in such an embodiment may be created as polonies.

As shown in FIG. 11, the gel 156 may be cut to expose the location-specific molecules and the multiple-location molecules to a sample 159 (similar to the sample 100, 152, or 155, for example), which may be stamped on one side of the gel 156, and the embodiment of FIG. 11 may otherwise be similar to the embodiments described above.

In the embodiment of FIG. 11, a plurality of analytes become associated with respective location-specific markers, and a plurality of multiple-location markers become associated with respective location-specific markers, when the plurality of location-specific markers and the plurality of multiple-location markers are suspended in a gel.

In the embodiments described above, when an analyte becomes associated with a location-specific marker, the analyte and the location-specific marker are at a location associated with a location-specific-marker type of the location-specific marker. Also, in the embodiments described above, when a multiple-location marker becomes associated with a respective location-specific marker, the location-specific marker is at a location associated with a location-specific-marker type of the location-specific marker. Also, the embodiments described above involve transferring multiple-location markers to multiple locations as described above. However, alternative embodiments may differ, as described below for example.

### Alternative Involving Detaching of Sample Components

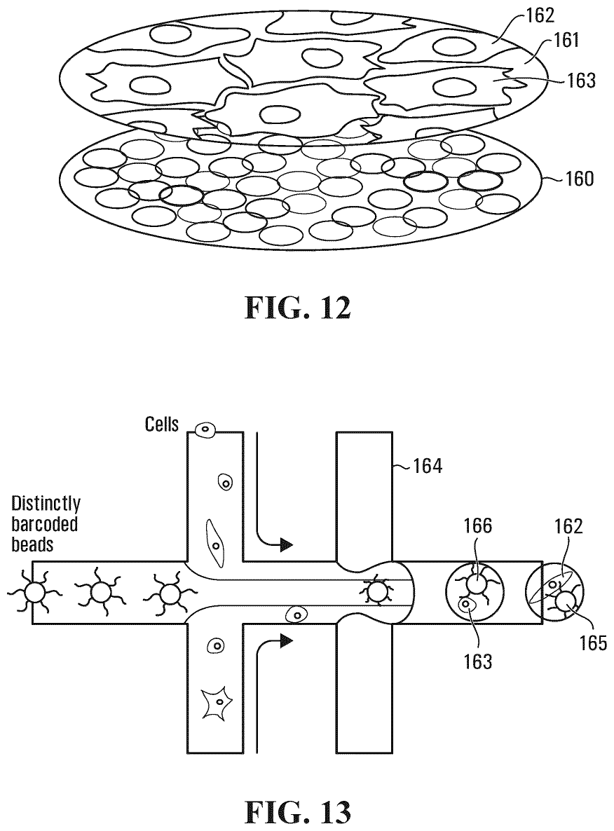

FIG. 12 and FIG. 13 schematically illustrate an example of a spatial-sequencing method according to another embodiment. As shown in FIG. 12, multiple-location molecules (similar to multiple-location molecules as described above, for example), each including a respective multiple-location marker, may be transferred from a surface of a paper disc 160 (that may be similar to the paper disc 136 as described above, for example) to a sample 161 (similar to the sample 100, 152, 155, or 159, for example). As a result, the multiple-location molecules are transferred to respective locations on a surface of the sample 161 or in the sample 161.

In the embodiment shown, the sample 161 includes animal cells such as animal cells 162 and 163, for example. When the multiple-location molecules are transferred to respective locations on the surface of or in the sample 161, the multiple-location molecules may bind to or otherwise become associated with animal cells such as the animal cells 162 and 163.

Referring to FIG. 13, after the multiple-location molecules are transferred to respective locations on the surface of or in the sample 161, animal cells of the sample 161 may be detached from each other. The animal cells of the sample 161 are therefore sample components of the sample 161. In alternative embodiments, sample components of at least one sample may differ and may not necessarily be animal cells.

After the animal cells of the sample 161 are detached from each other, the animal cells of the sample 161 (some of all of which are bound to or otherwise associated with one or more multiple-location molecules as described above) may be exposed to beads or other bodies (such as the beads or other bodies described above, for example) in a microfluidic chip 164. As shown in FIG. 13, for example, the animal cell 162 was exposed to, and binds to (or otherwise becomes associated with) a bead 165 in the microfluidic chip 164, and the animal cell 163 was exposed to, and binds to (or otherwise becomes associated with) a bead 166 in the microfluidic chip 164.

Like the beads 103 and 105 described above, RT primers may be on an outer surface the bead 165, RT primers may be on an outer surface the bead 166, at least some of the RT primers may be encoded with respective location-specific markers, the RT primers on the outer surface the bead 165 may each include location-specific markers of one location-specific-marker type, and the RT primers on the outer surface the bead 166 may each include location-specific markers of another location-specific-marker type. More generally, each of the beads (or other bodies) in the embodiment of FIG. 12 and FIG. 13 may have respective location-specific markers of a location-specific-marker type associated with the bead (or other body).

When an animal cell (or other sample component) is exposed to one of the beads (or other bodies),
1. the animal cell (or other sample component) may be an analyte bound to or otherwise associated with a location-specific-marker of the bead (or other body),
2. multiple-location markers of some or all of any multiple-location molecules bound to or otherwise associated with the cell (as described above) may become bound to or otherwise associated with respective location-specific-markers of the bead (or other body), and
3. a location
   a. associated with the bead (or other body),
   b. associated with the location-specific-markers of the bead (or other body), and
   c. associated with a location-specific-marker type of the location-specific-markers of the bead (or other body)
   may be a location of the animal cell (or other sample component) in the sample 161 before the animal cells of the sample 161 are detached from each other.

Therefore, in the embodiment of FIG. 12 and FIG. 13, location-specific-markers may be cell-specific markers (or markers specific to other sample components). Also, in the embodiment of FIG. 12 and FIG. 13, a plurality of analytes become associated with respective location-specific markers, and a plurality of multiple-location markers become associated with respective location-specific markers, after sample components are detached from each other.

The embodiment of FIG. 12 and FIG. 13 may otherwise be similar to the embodiments described above.

### Other Embodiments

Alternative embodiments may differ from the embodiments described above. For example, in some embodiments, a plurality of location-specific markers and a plurality of multiple-location markers may be distributed in three dimensions in at least one sample. In such embodiments, a location associated with a location-specific-marker type may be a 3D location of location-specific markers of the plurality of location-specific markers and of the location-specific-marker type in the at least one sample.

Also, other embodiments may involve distribution of location-specific markers, of multiple-location markers, or both by one or more different methods that may involve polony diffusion, electromagnetic attraction, creation by organisms such as E. coli, or one or more other methods, for example.

### Other Documents

The following documents may also assist the reader.
1. Greenstreet et al., “The DNA-based global positioning system—a theoretical framework for large-scale spatial genomics”, posted Apr. 5, 2022 at https://doi.org/10.1101/2022.03.22.485380
2. Kijima et al., “A universal sequencing read interpreter”, posted Apr. 16, 2022 at https://doi.org/10.1101/2022.04.16.488535 and published in Science Advances, vol. 9, issue 1 on Jan. 4, 2023 (doi: 10.1126/sciadv.add2793)

### CONCLUSION

In embodiments such as those described herein, positions in physical space of location-specific markers need not be known before the location-specific markers bind to or otherwise become associated with analytes. Rather, in embodiments such as those described herein, positions in physical space of analytes bound to or otherwise associated with location-specific markers may be determined by determining positions of locations as described above, for example.

Therefore, embodiments such as those described herein may permit spatial sequencing of larger samples when compared to other spatial-sequencing methods, or embodiments such as those described herein may otherwise improve spatial sequencing in scale, resolution, efficiency, effectiveness, or two or more thereof when compared to other spatial-sequencing methods. For example, embodiments such as those described herein may permit efficient spatial sequencing of a tumor, a portion of an animal organ, an entire animal organ, a portion of an animal, an entire animal, or another sample, that may be as large as 10,000 square millimeters (mm²) or larger, for example, with a relatively high resolution (such as a resolution from about 10 microns to about 30 microns, or a resolution at an animal-cell level or higher, for example).

Headings in this Detailed Description section are intended for illustration only and are not intended to limit the scope of the disclosure.

Further, although specific embodiments have been described and illustrated, such embodiments should be considered illustrative only and not as limiting the invention as construed according to the accompanying claims.

1. A spatial-sequencing method comprising:
causing a plurality of analytes from at least one sample to become associated with, at least, respective location-specific markers of a plurality of location-specific markers; and
causing a plurality of multiple-location markers to become associated with, at least, respective location-specific markers of the plurality of location-specific markers;
wherein each analyte of the plurality of analytes is of a respective analyte type of a plurality of different analyte types;
wherein each location-specific marker of the plurality of location-specific markers is associated with a respective location of a plurality of different locations relative to the sample;
wherein each location-specific marker of the plurality of location-specific markers is of a respective location-specific-marker type, associated with the location associated with the location-specific marker, of a plurality of different location-specific-marker types;
wherein each multiple-location marker of the plurality of multiple-location markers is of a respective multiple-location-marker type of a plurality of different multiple-location-marker types;
wherein causing the plurality of multiple-location markers to become associated with the respective location-specific markers comprises, for each multiple-location-marker type of the plurality of different multiple-location-marker types, causing multiple-location markers of the plurality of multiple-location markers and of the multiple-location-marker type to become associated with the respective location-specific markers associated with multiple locations of the plurality of the locations and associated with the multiple-location-marker type; and
wherein causing the plurality of multiple-location markers to become associated with the respective location-specific markers comprises, for each multiple-location-marker type of the plurality of different multiple-location-marker types, and for each location of the plurality of the locations and associated with the multiple-location-marker type, causing the multiple-location markers of the plurality of multiple-location markers and of the multiple-location-marker type to become associated with the respective location-specific markers at the location in a respective abundance that is positively correlated with a relative proximity of the location to a reference location of the multiple-location-marker type.

2. The method of claim 1 wherein causing the plurality of analytes to become associated with the respective location-specific markers comprises causing the plurality of analytes to bind to the respective location-specific markers.

3. The method of claim 1 wherein causing the plurality of multiple-location markers to become associated with the respective location-specific markers comprises causing the plurality of multiple-location markers to bind to the respective location-specific markers.

4-26. (canceled)

27. The method of claim 1 wherein each location-specific marker of the plurality of location-specific markers comprises a nucleotide sequence, and wherein the plurality of different location-specific-marker types is a plurality of different nucleotide sequences.

28. The method of claim 1 wherein each multiple-location marker of the plurality of multiple-location markers comprises a nucleotide sequence, and wherein the plurality of different multiple-location-marker types is a plurality of different nucleotide sequences.

29-30. (canceled)

31. The method of claim 1 wherein, for each multiple-location-marker type of the plurality of different multiple-location-marker types, the reference location of the multiple-location-marker type is a source of the multiple-location markers of the plurality of multiple-location markers and of the multiple-location-marker type.

32-38. (canceled)

39. The method of claim 1 wherein, when the plurality of analytes become associated with the respective location-specific markers, and when the plurality of multiple-location markers become associated with the respective location-specific markers, each location-specific marker of the plurality of location-specific markers is bound to a respective body of a plurality of bodies, each body of the plurality of bodies associated with a respective different location of the plurality of the locations.

40. The method of claim 39 wherein each body of the plurality of bodies comprises a bead.

41-49. (canceled)

50. The method of claim 1 wherein, when the plurality of analytes become associated with the respective location-specific markers, each location-specific marker of the plurality of location-specific markers is at the respective location of the plurality of different locations.

51-53. (canceled)

54. The method of claim 1 wherein:
the sample comprises a plurality of sample components;
the method further comprises detaching the plurality of sample components such that each sample component of the plurality of sample components is detached from each other sample component of the plurality of sample components; and
the plurality of analytes become associated with the respective location-specific markers of the plurality of location-specific markers after each sample component of the plurality of sample components is detached from each other sample component of the plurality of sample components.

55-57. (canceled)

58. The method of claim 1 wherein causing the plurality of multiple-location markers to become associated with the respective location-specific markers comprises causing the plurality of multiple-location markers to be transferred from at least one surface to the respective location-specific markers of the plurality of location-specific markers.

59. The method of claim 1 wherein causing the plurality of multiple-location markers to become associated with the respective location-specific markers comprises, for each multiple-location-marker type of the plurality of different multiple-location-marker types, causing the multiple-location markers of the plurality of multiple-location markers and of the multiple-location-marker type to be transferred from at least one surface to the multiple locations of the plurality of the locations and associated with the multiple-location-marker type.

60. The method of claim 1 wherein causing the plurality of multiple-location markers to become associated with the respective location-specific markers comprises, for each multiple-location-marker type of the plurality of different multiple-location-marker types, causing the multiple-location markers of the plurality of multiple-location markers and of the multiple-location-marker type to propagate, from a source of the multiple-location markers of the plurality of multiple-location markers and of the multiple-location-marker type, to the multiple locations of the plurality of the locations and associated with the multiple-location-marker type.

61. The method of claim 1 further comprising determining, at least, a multiple-location-marker estimation, for each location-specific-marker type of the plurality of different location-specific-marker types and for each multiple-location-marker type of the plurality of different multiple-location-marker types, of an estimated number of location-specific markers, of the plurality of location-specific markers and of the location-specific-marker type, associated with respective multiple-location markers of the plurality of multiple-location markers and of the multiple-location-marker type.

62. The method of claim 61 further comprising, in response to at least the multiple-location-marker estimations, identifying a position of the respective location associated with each location-specific-marker type of the plurality of different location-specific-marker types.

63. (canceled)

64. The method of claim 62 wherein identifying the position of the respective location associated with each location-specific-marker type of the plurality of different location-specific-marker types comprises dimensionality reduction.

65. The method of claim 64 wherein:
identifying the position of the respective location associated with each location-specific-marker type of the plurality of different location-specific-marker types comprises dimensionality reduction; and
the dimensionality reduction comprises, for each location-specific-marker type, identifying at least a manifold embedded in the estimated numbers of location-specific markers of the plurality of location-specific markers and of the location-specific-marker type.

66-67. (canceled)

68. The method of claim 64 wherein the dimensionality reduction comprises manifold learning.

69. (canceled)

70. The method of claim 1 further comprising determining, at least, a location-analyte estimation, for each location-specific-marker type of the plurality of different location-specific-marker types and for each analyte type of the plurality of different analyte types, of an estimated number of location-specific markers, of the plurality of location-specific markers and of the location-specific-marker type, associated with respective analytes of the analyte type.

71. The method of claim 1 wherein causing the plurality of multiple-location markers to become associated with the respective location-specific markers comprises, for each multiple-location-marker type of the plurality of different multiple-location-marker types, causing multiple-location markers of the plurality of multiple-location markers and of the multiple-location-marker type to become associated with the respective location-specific markers associated with some but not all of the locations of the plurality of the locations and associated with the multiple-location-marker type.
